(19) United States
(12) Patent Application Publication
(10) Pub. No.: US 2011/0054242 A1
(43) Pub. Date: Mar. 3, 2011

(54) Induced Relaxation And Therapeutic Apparatus And Method

(76) Inventor: Eddie L. Bender, Bartlett (US)

(21) Appl. No.: 12/553,798

(22) Filed: Sep. 3, 2009

(51) Int. Cl. A61M 21/02

(52) U.S. Cl. 600/28; 600/27

(57) ABSTRACT

An apparatus and method for introducing multisensory stimuli. The apparatus includes an ergonomically contoured seating device, at least one vibrating acoustic device, at least one plate for dispersing vibration throughout the entire seating device, a rotatable mechanism for rotating the seating device.

### CROSS-REFERENCE TO RELATED APPLICATIONS

This utility patent application is related to co-filed U.S. patent application Ser. No. 12/553,773, filed on Sep. 3, 2009, the disclosure of which is incorporated herein, by reference.

### TECHNICAL FIELD OF THE INVENTION

The present invention is generally related to an apparatus and method for stimulating sensory perceptions, including visual, aural, tactile, temperature, and olfactory senses for purposes of mental and physical stimulation, therapy, and relaxation. More particularly, the present invention relates to multisensory apparatus for stimulating visual, aural, tactile, temperature, and olfactory senses through visual presentations, audio inputs, and olfactory inputs to induce a greater degree of relaxation in an individual for achieving mental and physical therapeutic results.

### BACKGROUND OF THE INVENTION

There are numerous techniques for providing sensory stimulation for purposes of therapy for the release of stress to improve body functioning. By way of example, basic audio systems can alter heart rate and biorhythm functioning or create a soothing effect on individuals. Such audio systems, therefore, have been used for the purposes of stimulating an individual's response to what is aurally received.

Basic visual systems, which include, without limitation, two or three dimensional images for more realistic visual presentations, multiple lighting and strobing effects of images for stimulating brainwaves, have also been used for the purposes of stimulating an individual's response to what is visually perceived. Such systems are typically limited to stimulating only certain senses by a professional to the participant requiring therapy. Indeed, many devices useful in sensory stimulation are not capable of delivering multiple stimuli. In at least one case, a system has been developed that utilizes multiple stimuli including sound, motion, temperature, and visual stimuli. Effective operation of such a system, however, requires a relatively highly skilled professional at the same location as the system. Among other things, this requirement can limit access to specific individuals who desire to receive such therapy, relaxation treatment. In addition, such systems are costly and complex to use. Thus, for at least the aforementioned reasons, there exists a need in the art for advanced apparatus and methods for introducing multiple stimuli to an individual desiring relaxation and therapeutic benefits.

### SUMMARY OF THE INVENTION

The present invention has been devised to provide an improved relaxation apparatus and method to effectively induce a person into a state of relaxation. In an embodiment, the multisensory stimulation system is configured so that an individual is physically relaxed while seated on the apparatus and physically stimulated.

To this end, in accordance with a broad aspect of the present invention, an apparatus in accordance with the present invention provides multisensory stimuli for inducing a relaxation response comprising a unitary reclining seating device ergonomically contoured to support the whole body of a participant who desires relaxation and therapeutic benefits, at least one vibration transducer for providing a vibratory stimulation to the seating device, at least one plate placed over the vibration transducer for dispersing vibration to the entire seating device, a base for supporting the seating device, a control device for controlling multisensory stimuli, a controller for the vibration transducer device, a unitary rod wherein a first end of the rod connects to the seating device, and a second end of the rod fixedly connects to the base, and a rotatable means operatively integrated at the first end of the rod for rotating the seating device to stimulate a gentle wave-like rocking motion in the seating device to promote vestibular stimulation. Stated another way, three dimensional motion is provided via multi-axis computer actuated motion whereby the movement can include pitch and roll or random programmed undulations. Moreover, the seating device can be moved in all directions or combinations of directions with regard to pitch and roll.

Preferably, the contoured seating device is employed in the form of a unitary reclining chair wherein a seat portion, a seatback portion tilted relative to the seat, and a leg rest portion tilted to the seat are in one unitary manner that is ergonomically designed for use. However, a person of ordinary skill in the art will also appreciate that the seating device can have a configuration similar to a bed.

It is contemplated that the seating device is made of a foam material to enhance comfort during use. The foam material is corrugated to ease pressure on the body of a person during use. It is contemplated that the materials used for structural supports, not to mention the contoured shape and thickness of the seating device, can vary from that shown as long as sufficient support is provided for seating surface to enhance an individual comfort during extended therapeutic and relaxation use.

A vibrating transducer is provided to send vibration signals to the seating device for tactile purposes or for adding massage therapy and stimulating sensory touch response for a user when the apparatus of the invention is in use. The vibration transducer, therefore, may be mounted below the contour seating device such that different audio outputs can be provided from essentially any exterior source of audio supply such as a conventional tape or disk player amplifier or the like. A plate is provided in between the vibration transducer and the seating device to cause vibration on the entire device.

It is also contemplated that the vibration can be localized to a certain body portion of the person by configuring/coupling/connecting a multiple of transducers in a vest, rather than providing the transducer in the device. Therefore, a person who desires relaxation in this manner can wear the vest so that the vibration transducers configured/coupled/connected inside of the vest vibrate only the upper body of the person. If the vest is utilized to localize vibration on the user, it is contemplated that headphones can be utilized for a separate audio stimulation. When headphones are used, therefore, separate audio outputs can be directly provided to the headphones from any exterior source of audio supply. It is also contemplated that sounds can be computer generated so that enhanced sounds can be varied to obtain different audio responses impressions to an individual utilizing the sensory stimulation system of the invention.

It is contemplated that the front and the back of the vest include a plurality of pockets. The pockets of the vest are necessarily sized to retain a plurality of transducers, which are necessary to cause vibration.

A rotatable mechanism is employed to rotate or rock the device to stimulate gentle wave-like rocking motion to promote vestibular stimulation to the user. The rotatable mechanism configured to the seating device has a capability of rocking the device back and forth, side-to-side, pivot, and rotate. It is contemplated that the device may be supported by a base with a unitary axis or rod connected therebetween to enable the device to rock back and forth, side-to-side, pivot, and rotate in clockwise or counter-clockwise directions. Stated another way, three dimensional motion is provided via multi-axis computer actuated motion whereby the movement can include pitch and roll or random programmed undulations. Moreover, the seating device can be moved in all directions or combinations of directions with regard to pitch and roll.

A monitor is mounted above the device (not shown in the drawings) so that the light and images therefrom are transmitted for viewing. It is contemplated that the monitor may be in the form of image projectors, including, but not limited to, television screens which are connected to an appropriate video input such as a video cassette, CD or DVD player so that prerecorded visual programs can be played through the monitor to a person utilizing the system while comfortably laying on the contoured seating device.

Control of the various elements of the invention may be accomplished through a control panel mounted to one side of the base or a handheld remote or wired controller. In some embodiments, computer actuated controls may be utilized to create an interaction delivery system such that pre-programming may be updated or changed depending upon a person's responses. Responses may be based on any combination of biofeedback sensors, neurofeedback sensors, electroencephalography (EEG), neurofeedback (NFB), or other conscious or subconscious inputs to thereby enhance sensory simulation utilizing a variety of visual, audio, and olfactory sensory actuators.

To enable the person resting on the device to be quickly led to relaxation, the vibration applied from the vibration transducer to the seating device and then to the body of the person is preferably of a kind that the vibration produced by the acoustic device acts directly on the whole body of the person and wherein the seating device is movable in a direction conforming to a direction of propagation of vibrations transmitted by the vibrating acoustic device to the person.

The seating device is vibrated in the horizontal (leftwards and rightwards) direction perpendicular to the longitudinal sense of the body of the person desiring relaxation. The vibration produced by the acoustic device may also be applied to the body of the person in any desired manner and in any desired acceleration mode. By way of example, where the seating device comprises a reclining chair of the type referred to hereinbefore, (i.e., that having a tiltable seat back and tiltable leg rest), the reclining chair as a whole may be vibrated in one or a combination of any desired directions and locations.

It is contemplated that change in vibratory signal and/or effective acceleration may be automatically accomplished either according to the length of time passed or manually by the user. Where one or both of the vibratory signal and the effective acceleration are desired to be changed or adjusted according to the length of time passed or the number of times of application of the vibration, this can be accomplished by the use of a timer or a presetting device. Design may be made so that one or both of the vibratory signal and the effective acceleration can be gradually reduced according to the length of time passed or the number of times of application of the vibration, so that the person on the device can be smoothly led to relaxation. It is also contemplated that the vibratory frequency is variable. It is further contemplated that the acceleration is variable. It is also contemplated that both the frequency and the acceleration are varied according to a pattern of vibration applied to the user.

Utilizing the methodology of the present invention for inducting a relaxation response, an individual is initially seated on a seating device in a zero gravity position so that the monitor is comfortably positioned relative to the individual.

To stimulate the various senses therefor, once the monitor is correctly positioned, the programmer, a pre-programmed computer or a media playback device using pre-recorded media may be activated to control various sensory stimulation elements. These elements include, but not limited to, visual, aural, tactile, temperature, and olfactory sensory. Depending upon the types of images being projected, different responses are induced in the individual. To further enhance inducement of the relaxation response, the user reclined in a zero gravity position on an ergonomically contoured seating device receives vestibular stimulation through the device undergoing gentle wave-like rocking motion. Stated another way, three dimensional motion is provided via multi-axis computer actuated motion whereby the movement can include pitch and roll or random programmed undulations. It is contemplated that the movement of the device promotes stimulation of cells, among other things, thereby increases blood flow and promotes heightened physical relaxation.

During the visual display and vestibular stimulation, further sensory stimulation may be effected by activation of the speakers or headphones through input from a sound system. Activation of the speakers or headphones through input from a sound system may be accomplished either manually, by pre-programmed computer input or by playback of prerecorded media queues. Dispersion of sound is caused by the transducers incorporated therein the speakers or vest, and creates vibration on the device or directly on the person thereby further stimulating the whole body of the user reclined on the device.

Although not disclosed in the preferred embodiment shown in the drawings, further modifications could be made to the system such as to reorient the seating device and/or the sensory stimulating system to generate further sensory responses of a person during the use of the system of the invention.

It is also contemplated that various other sensory stimulants and options may be incorporated into the device and system to achieve optimal stimulation response for each individual to induce a relaxation response. By way of example, during the visual, vestibular stimulation, an aroma system may be activated to further a sensory stimulation of a user. Activation of the aroma system may be accomplished either manually or by pre-programmed computer input. An ordinary skill in the art will understand the intricacies of utilizing the aroma system for stimulating sensory response.

It is further contemplated that in order to achieve optimal stimulation response for therapy and inducing relaxation, a heating means and a cooling means, controlled via a processor, may be incorporated in any one of the back, the seat, and the leg rest of the seating device.

It is desired that, in an embodiment to induce relaxation, the system allows an individual to be subjected to selected combinations of visual, audio, tactile, olfactory, and other stimulates in various combinations to affect beneficial mental and physical response for an individual using the system.

It is also desired that, in an embodiment in accordance with the present invention, a system and method is provided for inducing a relaxation response wherein the system can be adjusted to suit substantially any individual and wherein the system may be controlled either by a separate operator, by pre-programmed inputs or by interaction feedback or combinations thereof in order to achieve optimal stimulation response for each individual.

### BRIEF DESCRIPTION OF THE DRAWINGS

The invention may be better understood with reference to the following drawings. The components in the drawings are not necessarily to scale, emphasis instead being placed upon illustrating the principles of the invention. In the drawings, like reference numbers designate corresponding parts throughout.

FIG. 1 is a perspective partial fragmentary view of a contour seating device in accordance with the present invention;

FIG. 2 is a side view of the contour seating device of FIG. 1;

FIG. 3 is a simplified functional block diagram of a controller for operating the seating device of FIG. 1 or the vest of FIG. 5;

FIG. 4 is a simplified functional block diagram of the controller of FIG. 3;

FIG. 5 is a front plan view of a vest in accordance with the present invention;

FIG. 6 is a back plan view of the vest of FIG. 5;

FIG. 7 is a plan view of a mattress topper or cover in accordance with the present invention;

FIG. 8 is a side view of an alternative embodiment of a contour seating device in accordance with the present invention;

FIG. 9 is a perspective view of the base of FIG. 8 wherein the unitary reclining seat has been removed;

FIG. 10 is another embodiment of an apparatus in accordance with the present invention comprising a mattress lying on an adjustable platform; and,

FIG. 11 is an elevation view of the mattress of FIG. 10 lying on a planar surface.

### DETAILED DESCRIPTION OF THE INVENTION

The following descriptions of detailed embodiments are for exemplifying the principles and advantages of the inventions. They are not to be taken in any way as limitations on the scope of the inventions.

As used herein, the term “absolute relaxation” generally refers to an internal state of relaxed awareness in a person that is serene, boundless, inner milieu, going beyond the mind, going into silence, and entering a state of pure nothingness. Also, the term “induced relaxation” generally refers to the process of bringing an individual to a state of absolute relaxation by unconsciously removing anxiety and stress by accelerating the relaxation response through the use of an automated program that stimulates the human senses such as: touch, hearing, sight, and smell. Accordingly, the term “relaxation response” is generally defined as a physical state that is the opposite of the fight-or-flight response when the body is no longer in perceived danger, and the autonomic nervous system functioning returns to normal. The relaxation response results in reduced blood pressure and blood glucose levels, and lower breathing and heart rates allowing the body to release stress and anxiety, and thus enter a relaxed state.

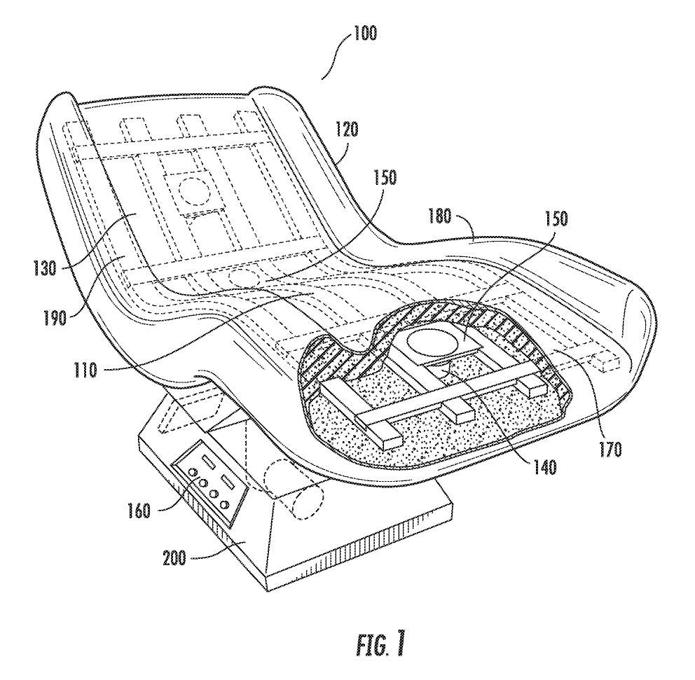

The contour seating device 100 in accordance with the present invention generally comprises a unitary reclining seat 120 for supporting a person desiring relaxation in his or her entirety, at least one vibration transducer 140 for providing a vibratory stimulus to the person through the seat 120 and a control device 160 for controlling the vibration transducer 140. FIG. 1 illustrates the seating device 100 in a reclined zero gravity position. The seating device 100 shown therein comprises a base 200 accommodating therein a control device 160 including a gear assembly 220 as a rotating means for rotating the seating device 100 in clockwise or counter clockwise directions, a motor assembly 260, a unitary reclining seat 120 mounted atop the box base 200 pre-configured in a zero gravity position, at least one vibration transducer 140 configured in the seating device 100, at least one metal, metal alloy, or plastic plate 150 provided in between the vibrating acoustic device 140, and the seating device 100, and a pair of arm rests or side bumpers 180, 190 extending entirely along the side of the seating device 100.

As illustrated in FIG. 1, the seating device 100 is contoured in the form of a unitary reclining chair wherein a seat portion 110, a seatback portion 130 tilted relative to the seat portion 110, and a leg rest portion 170 tilted to the seat portion 110 are in one unitary manner that is ergonomically predesigned for use. A person ordinary skill in the art will also appreciate that the seating device can have a configuration similar to a bed.

The seating device of FIG. 1 includes a frame with foam material attached thereto by glue or the like for enhancing comfort during use. However, it is contemplated that the materials used for structural supports, not to mention the contoured shape and thickness of the seating device, can vary from that shown as long as sufficient support is provided for seating surface to enhance an individual comfort during extended therapeutic and relaxation use. In an embodiment, the frame can consist of one or more pieces of plywood that are conformed by conventional means to the desired shape.

The seating device of FIG. 1 is operatively supported by the base 200. A single rod 210 is provided therebetween to connect the seating device 100 to the base box 200 for providing movement to the seat. A first end 211 of the rod is affixed to the seating device 100 and a second end 212 of the rod 210 is fixedly affixed to the base box. The rotation of the seating device 100 is possible by an actuating mechanism 220 connected to the second end 212 of the rod 210. It is contemplated that a person ordinary skill in the art will understand that other mechanisms are available to enable the seating device 100 to rotate, rock, or move about in a desired orientation relative to the base box 200. Preferably, but not necessarily, the rotation of the chair can be selected in either a counterclockwise or clockwise direction. However, in an alternative embodiment, the chair can be moved in all directions or combinations of directions with regard to pitch and roll.

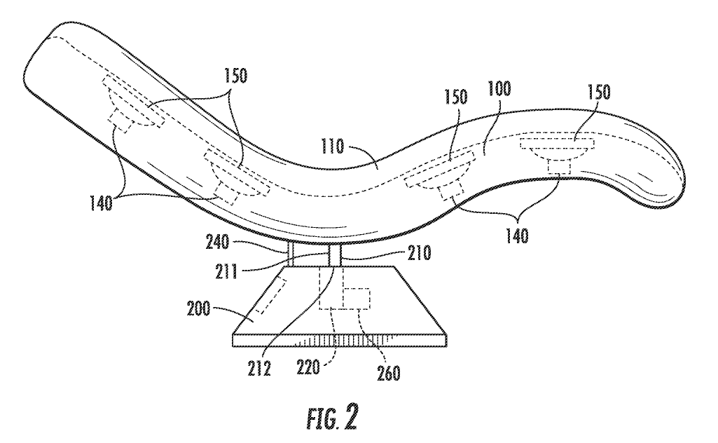

As shown in FIG. 2, connected to the chair and the box is a stabilizer linkage 240 that prevents changes in yaw of the chair, but still allows for changes in pitch and roll of the chair. In an embodiment, the stabilizer linkage can include a pair of parallel metal beams secured at one end to the base box with a sliding guide pin positioned therebetween and attached to the chair. As such, the guide pin can reciprocate between the beams, to accommodate changes in pitch of the chair, and also turn between the beams to accommodate changes in roll of the chair. Stated another way, three dimensional motion is provided via multi-axis computer actuated motion wherein the movement can include pitch and roll or random programmed undulations.

Turning back to FIG. 1, the vibration transducer 140 is housed below the seating device 100. As will be appreciated by those having skill in the art, the transducer can be a tactile transducer or bass shaker constructed using the principle that low bass frequencies can be felt as well as heard. As such, the shaker transmits low-frequency vibrations into various surfaces so that they can be felt by people. In an embodiment, this vibration transducer 140 is so configured and so wired as to vibrate the seating device 100 in its entirety including not only the seatback portion 130, but also the leg rest portion 170 during activation of the vibration transducer 140 by frequencies transmitted from the controlling device 160. Having the plate 150 configured therebetween the vibration transducer 140 and the seating device 100 allows for the vibration to affect the entire seating device 100. Accordingly, when an individual desiring relaxation is seated on the seat portion 110 of the seating device 100 with his or her back resting on the seatback portion 130 and with his or her legs resting on the leg rest portion 170, the seat occupant of the seating device can be vibrated in his or her entirety.

The vibration transducer 140 is of a type capable of providing the seating device 100 with vibrations of a low frequency and at an effective acceleration. For the purpose of the present invention, a specific mechanism for generating the vibration is immaterial so far as the vibration transducer 140 satisfies the frequency and/or acceleration in order to disperse vibration on an entire seating device 100. In an embodiment, however, the transducers 140 can have a resistance of 2 to 4 ohms and a power rating of 50 Watts.

The vibration transducer 140 has been shown as accomplishing a uniform vibration in the seating device 100 in its entirety. However, if desired, a localized vibration may be applied to only a portion of the body of the seat occupant such as, for example, back, or legs of the seat occupant.

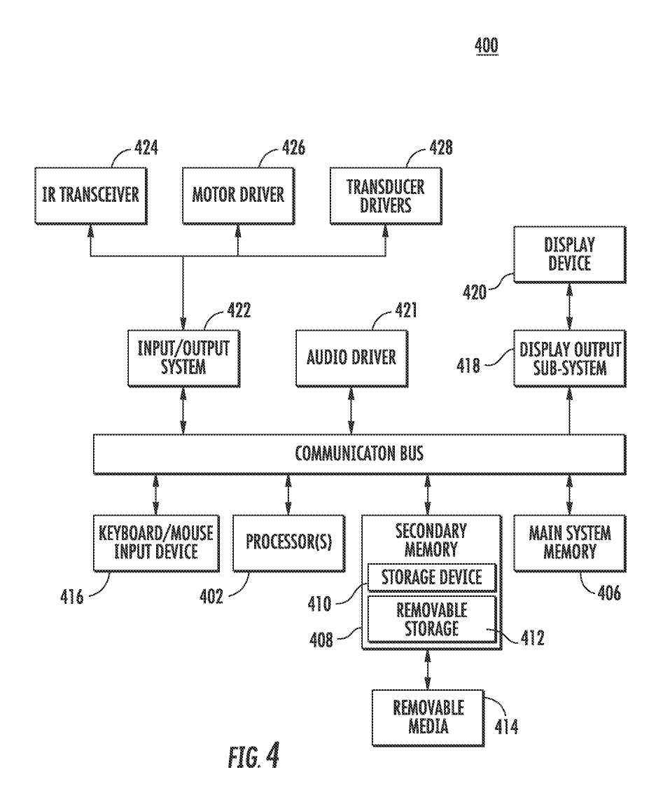

The control device 160 for controlling the vibration transducer 140 may be conveniently employed in the form of a micro computer. The control device 160 can also control the vibration applied to the seating device 100 by mechanical means (not shown in drawings). It is also contemplated that the control device 160 controls the heating means, the cooling means, and audio, visual, olfactory, and tactile stimuli for providing the combination of stimulus discussed above. It will be understood by a person ordinary skill in the art that the control device 160 may be programmed to permit the user to operate the seating device of the present invention in a manner as shown in the flowchart of FIG. 4.

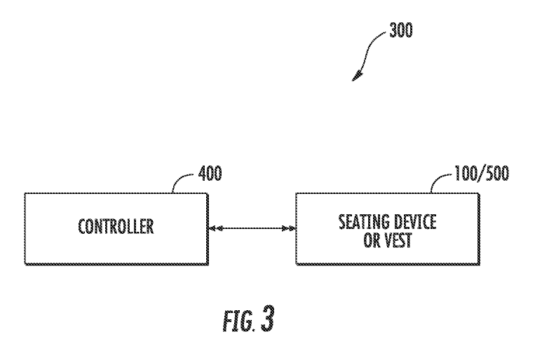

With respect to FIG. 3, an illustrational diagram is shown with respect to the interactive system 300 for operating the seating device 100 with computer system of the invention discussed below in FIG. 4. While the exemplary system of FIG. 3 shows wired coupling between the computer system 400 and the seating device 100, one of ordinary skill in the art will recognize that various media and other communication means can be used to provide the various communicable couplings to carry out the present invention. For example, the interactive system 300 may include the use of biofeedback sensors, neurofeedback sensors, or a combination thereof such as but not limited to body temperature sensors, and possibly a voice recognition system as well as various other input devices to both subconsciously and consciously manipulate or alter the programmed control of multisensory stimulation being applied to the seating device 100.

As will be discussed in more detail below, the computer system 400 is operable to distribute the preprogrammed output signal to the seating device 100, one or more of the vibration transducers 140, the display device 420, the motor driver 426, all depending on the type of the output signal necessary for implementing the present invention so that the visual, aural, tactile, temperature, and olfactory senses are stimulated to relax a person while sitting on the seating device 100. As such, the computer system 400 can be any microprocessor based device capable of communicating and issuing commands to one or more of the seating device 100, transducer 140, display device 420, audio driver 421, and motor driver 426. By way of example, in one exemplary embodiment of the present invention, one software program can be a sensory program that directs or interactively delivers the introduction of various sensory inputs to a participant via one or more of the seating device 100, transducer 140, display device 420, audio driver 421, and motor driver 426.

In an embodiment in accordance with the present invention, programs can be stored on the computer system corresponding to one or more relaxation or therapeutic sessions for selection by a user. Each session can include a pre-selected audio presentation, video presentation, vibration duration time, and vibration intensity. For instance, a session can include a soft music presentation along with an ocean view video presentation, mild vibration intensity, and a program length of about thirty (30) minutes. The vibration intensity can also correspond with the intensity of the music presentation. For instance, as the tone of the music increases, so does the vibration intensity. Likewise, as the tone of the music decreases, so does the vibration intensity. As such, the vibration intensity is responsive to the audio track or a low base track of the music presentation.

The session choices for selection by a user can be provided on a touch screen, video display or the like. Moreover, a session can provide for guided hypnotic suggestion(s) or mediation. For instance, the audio presentation can include a prerecorded voice track or voice over with suggestions for the user to listen to. The suggestions can be tailored for the user and include, for example, tracks directed to overcoming drug addiction, alcoholism, smoking, depression or other undesirable traits, characteristics or behavioral problems.

FIG. 4 depicts an exemplary embodiment of a block diagram 400 illustrating an exemplary embodiment of a computer system or controller 400 used in conjunction with chair 100. The controller in accordance with the present invention (or any part(s) or function(s) thereof) may be implemented using hardware, software, firmware, or a combination thereof and may be implemented in one or more computer systems or other processing systems. In fact, in one exemplary embodiment, the invention may be directed toward one or more computer systems capable of carrying out the functionality described herein. An example of a computer system 400 is shown in FIG. 4, depicting an exemplary embodiment of a block diagram of an exemplary computer system useful for implementing the present invention. Specifically, FIG. 4 illustrates an example computer 400, which in an exemplary embodiment may be, e.g., (but not limited to) a personal computer (PC) system running an operating system such as, e.g., (but not limited to) MICROSOFT WINDOWS available from MICROSOFT Corporation of Redmond, Wash., U.S.A., or Mac/OS from APPLE Corporation of Cupertino, Calif., U.S.A. However, the invention may not be limited to these platforms. Instead, the invention may be implemented on any appropriate computer system running any appropriate operating system. In one exemplary embodiment, the present invention may be implemented on a computer system operating as discussed herein. An exemplary computer system, computer 400 is shown in FIG. 4.

The computer system 400 may include one or more processors, such as, e.g., but not limited to, processor(s) 402. The processor(s) 402 may be coupled or connected to a communication infrastructure 404 (e.g., but not limited to, a communications bus, a backplane, a mother board, a cross-over bar, or network, etc.). Various exemplary software embodiments may be described in terms of this exemplary computer system. After reading this description, it will become apparent to a person skilled in the relevant art(s) how to implement the invention using other computer systems and/or architectures.

Computer system 400 may include a display interface 418 that may forward, e.g., but not limited to, graphics, text, and other data, etc., from the communication infrastructure 404 (or from a frame buffer, etc., not shown) for display on the display unit 420. Preferably, but not necessarily, the display 420 is a flat screen monitor that depicts operational user menus and scenic video clips or presentations to assist in relaxing and stimulating the person on the chair 100. The display 420 can be mounted to a ceiling or other suitable structure for placing the display at an angle above the user of the chair 100. In use, it is preferred that the chair is operated in a dark or dimly lit room such that the attention of the user is drawn of the presentation provided on the display 420.

The computer system 400 may also include, e.g., but may not be limited to, a main memory 406, which may include, e.g., but not limited to, random access memory (RAM), and a secondary memory 408, etc. The secondary memory 408 may include, for example, (but not limited to) a storage device 410 such as, e.g., but not limited to, a hard disk drive and/or a removable storage drive 412, representing, e.g., but not limited to, a floppy diskette drive, a magnetic tape drive, an optical disk drive, a compact disk drive CD-ROM, a magneto-optical (MO) drive, a digital versatile disk (DVD), etc. The removable storage drive 412 may, e.g., but not limited to, read from and/or write to a removable storage unit 414 in a well known manner. Removable storage media unit 414, may also be called a program storage device or a computer program product, and may represent, e.g., but not limited to, a floppy disk, magnetic tape, optical disk, CD-ROM disk, a MO device, a DVD disk, etc. which may be read from and written to by removable storage device 412. As will be appreciated, the removable storage unit 414 may include a computer usable storage medium having stored therein computer software and/or data. As indicated previously, the memories may include scenic video clips or presentations to assist in relaxing and stimulating the person on the chair 100. This clips or presentations may include an audio portion comprising music or other relaxing or stimulating sounds. These audio portions may be provided to the audio driver 421 wherein they are audibly reproduced by headphones or one or more speakers. The headphones can be placed on the client or user of the chair 100 wherein, preferably, but not necessarily, the user can control the volume of the volume of the reproduced should via a convention knob on the headphones. Preferably, but not necessarily, the magnitude of the vibrations generated by the transducers 140 is proportional to the audio signal provided by driver 421.

In alternative exemplary embodiments, secondary memory 408 may include other similar devices for allowing computer programs or other instructions to be loaded into computer system 400. Such devices may include, for example, a removable storage unit 414 and an interface (not labeled). Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units 414 and interfaces, which may allow software and data to be transferred from the removable storage unit 414 to computer system 400.

Computer 400 may also include, e.g., but not limited to, an input device 416 such as, e.g., (but not limited to) a mouse or other pointing device such as a digitizer, and a keyboard or other data entry device (none of which are labeled).

Computer 400 may also include, e.g., but not limited to, other output devices, such as, e.g., (but not limited to) display 420, and output subsystem display interface 418.

Computer 400 may also include, e.g., but not limited to, input/output (I/O) system 422 such as, e.g., (but not limited to) a communications interface, a cable and communications path, (all not shown) etc., as well as I/O devices 424, 426, 428, for example.

The I/O system can include a multiplexer for controlling one or more (e.g., transducers 140) via driver 428. Further, the I/O system 422 can provide for receiving remote controller signals received from conventional IR transceiver 424, or the like. In an embodiment, but not necessarily, the diver 428 can be an Audio Power Amplifier by Dayton Audio, Springboro, Ohio, model number APA150.

In this document, the terms “computer program medium” and “computer readable medium” may be used to generally refer to media such as, e.g., but not limited to removable storage drive 414, a hard disk installed in storage device 410, and signals, etc. These computer program products may provide software to computer system 400. The invention may be directed to such computer program products.

In another exemplary embodiment, the invention may be directed to a computer program product comprising a computer readable medium having control logic (computer software) stored therein. The control logic, when executed by the processor 402, may cause the processor 402 to perform the functions of the invention as described herein. In another exemplary embodiment where the invention may be implemented using software, the software may be stored in a computer program product and loaded into computer system 400 using, e.g., but not limited to, removable storage drive 412, storage device 410 or communications interface, etc. The control logic (software), when executed by the processor 402, may cause the processor 402 to perform the functions of the invention as described herein. The computer software may run as a standalone software application program running atop an operating system, or may be integrated into the operating system.

In yet another embodiment, the invention may be implemented primarily in hardware using, for example, but not limited to, hardware components such as application specific integrated circuits (ASICs), or one or more state machines, etc. Implementation of the hardware state machine so as to perform the functions described herein will be apparent to persons skilled in the relevant art(s).

In another exemplary embodiment, the invention may be implemented primarily in firmware.

In yet another exemplary embodiment, the invention may be implemented using a combination of any of, e.g., but not limited to, hardware, firmware, and software, etc.

Exemplary embodiments of the invention may also be implemented as instructions stored on a machine-readable medium, which may be read and executed by a computing platform to perform the operations described herein. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other form of propagated signals (e.g., carrier waves, infrared signals, digital signals, etc.), and others.

The exemplary embodiment of the present invention makes reference to wired, or wireless networks. Wired networks include any of a wide variety of well known means for coupling voice and data communications devices together. A brief discussion of various exemplary wireless network technologies that may be used to implement the embodiments of the present invention now are discussed. The examples are non-limited. Exemplary wireless network types may include, e.g., but not limited to, code division multiple access (CDMA), spread spectrum wireless, orthogonal frequency division multiplexing (OFDM), 1G, 2G, 3G wireless, Bluetooth, Infrared Data Association (IrDA), shared wireless access protocol (SWAP), “wireless fidelity” (Wi-Fi), WIMAX, and other IEEE standard 802.11-compliant wireless local area network (LAN), 802.16-compliant wide area network (WAN), and ultrawideband (UWB), etc.

Bluetooth is an emerging wireless technology promising to unify several wireless technologies for use in low power radio frequency (RF) networks.

IrDA is a standard method for devices to communicate using infrared light pulses, as promulgated by the Infrared Data Association from which the standard gets its name. Since IrDA devices use infrared light, they may depend on being in line of sight with each other.

The exemplary embodiments of the present invention may make reference to WLANs. Examples of a WLAN may include a shared wireless access protocol (SWAP) developed by Home radio frequency (HomeRF), and wireless fidelity (Wi-Fi), a derivative of IEEE 802.11, advocated by the wireless ethernet compatibility alliance (WECA). The IEEE 802.11 wireless LAN standard refers to various technologies that adhere to one or more of various wireless LAN standards. An IEEE 802.11 compliant wireless LAN may comply with any of one or more of the various IEEE 802.11 wireless LAN standards including, e.g., but not limited to, wireless LANs compliant with IEEE std. 802.11a, b, d or g, such as, e.g., but not limited to, IEEE std. 802.11a, b, d and g, (including, e.g., but not limited to IEEE 802.11g-2003, etc.), etc.

In the foregoing description, the vibration acoustic device has been shown as accomplishing a uniform vibration in the seating device in its entirety. However, if desired, a person ordinary skill in the art will also understand that other means of producing the same vibration are available, e.g. by a mechanical means.

In another embodiment where the vibrating acoustic devices are not configured in the seating device, an individual desiring relaxation can wear a vest 500 that looks a lot like a fishing vest.

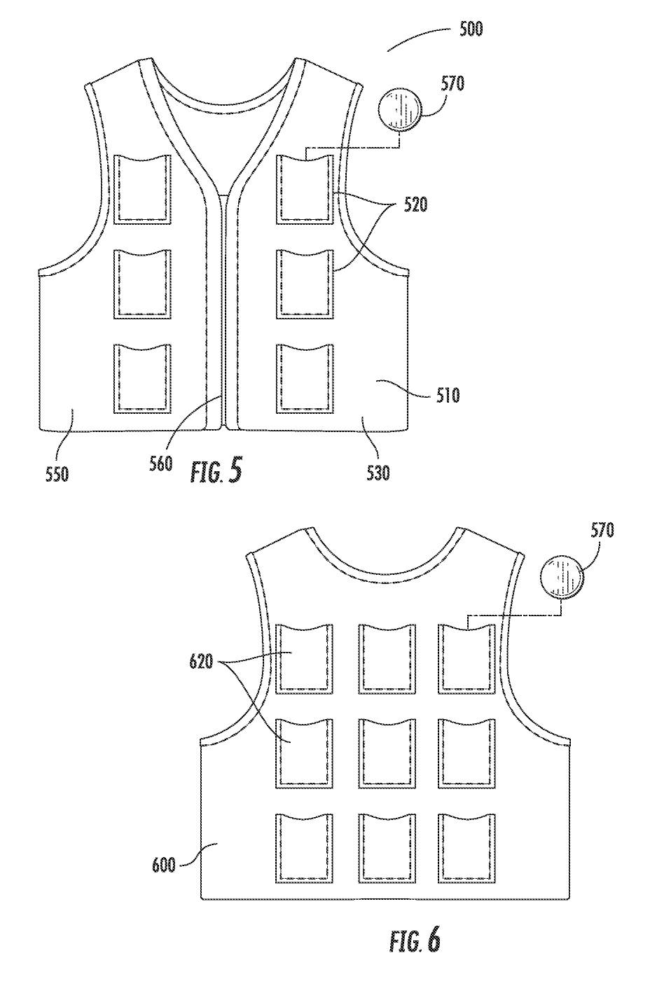

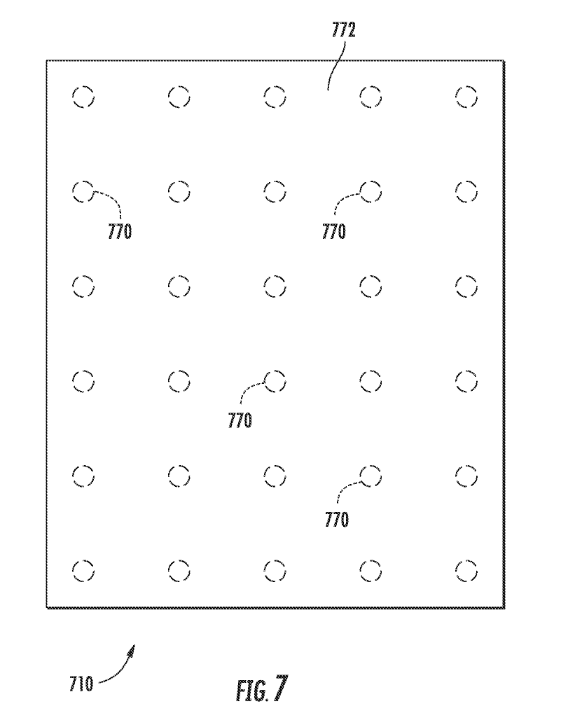

The vest 500 according to the present invention comprises a plurality of pockets 520. The front section 510 of the vest 500, as shown in FIG. 5, includes two side portions 530, 550. The two side portions 530, 550 are interconnected by zippered portion 560. Each side portion includes a plurality of pockets 520 on the outside for holding vibrating transducer devices 570 that are about the size of a hockey puck, have a resistance of 2 ohms, and a power rating of 10 Watts. A rear section of the vest 600, as shown in FIG. 6, includes a plurality of pockets 620 on the outside for holding vibrating transducer devices 670.

Typically, pockets 520, 620 of FIGS. 5 and 6 may be constructed by cut and sewn fabric pieces. Alternatively, pockets 520, 620 may be molded. In addition, a zipper or other closure mechanism (such as snap, button, Velcro or magnet closure) and/or other fabric elements may be used to interconnect the two side portions 530, 550 of the vest 500.

In use, the vest 500, and in particular the transducers 570, are communicably coupled to the computer system 400 by wires or the like to carry out the present invention, as shown in FIG. 3. As such, the computer system 400 can directly issue a separate command to the vest 500 and bypass the seating device 100 to introduce tactile stimulation to a participant. The introduction of various other sensory inputs remains the same as before. For the purpose of this invention, therefore, it will become apparent to a person skilled in the relevant art(s) how to implement the invention using this or other computer systems and/or architectures coupled to the vest 500. Moreover, the vest can be used with a conventional headphone to provide the user with an audio presentation.

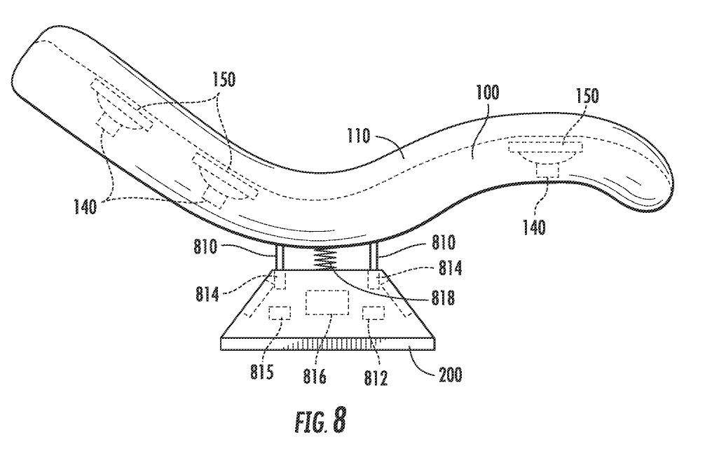

Turning to FIG. 7, one or more vibrating transducers 770 can be contained within the foam or other filling material 772 of a mattress topper or cover. 710 that lies on top or over a convention mattress (not shown) for a bed. Accordingly, the transducers 770 can be connected to the computer system of FIG. 4 by wires or other conventional means and operated similar to the vest of FIGS. 5-6.

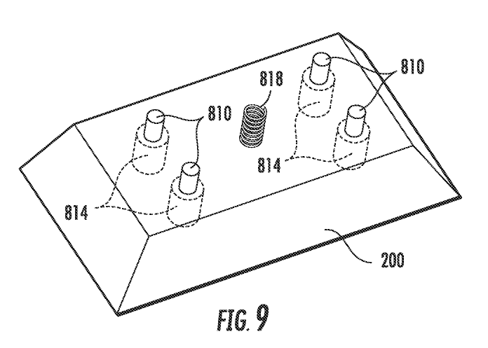

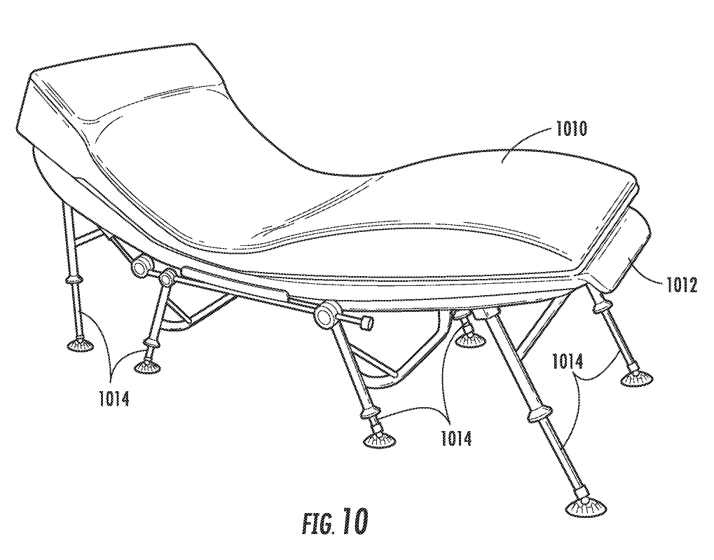

Turning to FIG. 8, a side view is provided of an alternative embodiment of a contour seating device in accordance with the present invention. Like that depicted in FIGS. 1 and 2, the seating device of FIG. 8 includes a seat or chair portion 110 and a base 200. However, movement of the seat 110 is caused by actuation of one or more shocks, pistons, or air bellows 810 coupled to both the seat and the base 200. Actuation of the cushions, pistons or bellows 810 can be controlled by conventional means such as by opening and closing valves 812 or operation of a digital pressure regulator for allowing a pressurized air, gas or liquid (i.e., a fluid), provided by pump 815 to enter and exit a air cushion or piston cylinder 814 associated with each device 810. Further, a bias member, such as a coiled tension spring 818, can be attached to the seat 110 and the base 200 for maintaining a compression force against the cushions or pistons 810 to ensure that they contract when fluid or pressure is released from the device 814.

The opening and closing of the valves 812 can be controlled by a controller 816. Accordingly, changes in pitch and roll of the seat 110 are responsive to commands from the controller 816 to open and close the valves 812. Preferably, like the embodiment depicted in FIGS. 1 and 2, the seat 110 of FIG. 8 is rotated via actuation of the pistons or air cushions to simulate a gentle wave-like rocking. Stated another way, three dimensional motion is provided via multi-axis computer actuated motion whereby the movement can include pitch and roll or random programmed undulations.

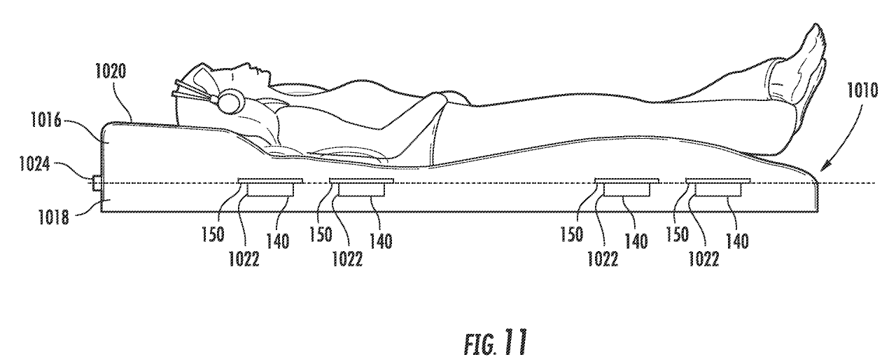

In FIGS. 10 and 11, a mattress in accordance with the present invention is depicted comprising a neoprene covered foam body with a plurality of transducers embedded within the foam body. In FIG. 10, the mattress 1010 is supported by an adjustable platform such as an adjustable cot 1012 having legs 1014 that can be lengthened or shorten, as desired.

As shown in FIG. 11, the foam body 1010 can include an upper portion 1016 and a lower portion 1018 that can be attached together by glue or other conventional means. The upper portion 1016 of the body 1010 can be constructed of a foam material and includes an upper surface 1020 that is contoured to comfortably support an adult human body lying on his or her back.

The lower portion 1018 is also constructed of a foam material that can be the same, or different from, the foam material used to construct the upper portion 1016. The lower portion 1018 is generally rectangular in vertical and horizontal cross-section and includes a plurality of cavities 1022 for receiving the transducers 140. In an embodiment, but not necessarily, the transducers 140 are mounted in spaced parallel relationship to the longitudinal axis of symmetry for the lower portion 1018 of the body 1010. In addition, a plastic rectangular plate 150 is preferably mounted over the opening to each cavity 1022 and is attached to the transducer 140 within the cavity. Accordingly, the plates 150 are sandwiched between the lower portion 1018 and the upper portion 1016.

The transducers 140 are electrically coupled, via wires, to an electrical connector 1024. In turn, the electrical connector 1024 can be connected by conventional means to the transducer drivers 428 of FIG. 4.

It is also contemplated, although not shown in the drawings, that an individual may be monitored in order to determine what effects and responses the individual undergoes in response to presented multisensory stimulation. For example, the present invention may be used in cooperation with biofeedback, neurofeedback, or a system using a combination thereof such that sensors, e.g., brainwave sensors, are applied to the person undergoing stimulation so as to provide feedback information with respect to body temperature, pulse rate, blood pressure, and related bodily functioning. It is further contemplated that depending on the biofeedback and/or neurofeedback of the person undergoing stimulation, a combination of motion, visual, audio, olfactory, and other stimuli can be varied. A person ordinary skill in the art understands that there are various biofeedback and/or neurofeedback systems which may be suitable for use with the present invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still co-operate or interact with each other.

An algorithm is described directly or indirectly herein, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, as apparent from the following discussions, it is appreciated that throughout the specification discussions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments of the present invention may include apparatuses for performing the operations herein. An apparatus may be specially constructed for the desired purposes, or it may comprise a general purpose device selectively activated or reconfigured by a program stored in the device.

Embodiments of the invention may be implemented in one or a combination of hardware, firmware, and software. Embodiments of the invention may also be implemented as instructions stored on a machine-readable medium, which may be read and executed by a computing platform to perform the operations described herein. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other form of propagated signals (e.g., carrier waves, infrared signals, digital signals, etc.), and others.

Computer programs (also called computer control logic), may include object oriented computer programs, and may be stored in main memory 406 and/or the secondary memory 408 and/or removable storage media units 414, also called computer program products. Such computer programs, when executed, may enable the computer system 400 to perform the features of the present invention as discussed herein. In particular, the computer programs, when executed, may enable the processor 402 to provide a method to resolve conflicts during data synchronization according to an exemplary embodiment of the present invention. Accordingly, such computer programs may represent controllers of the computer system 400.

The foregoing also describes only some embodiments of the present invention and modifications, obvious to those skilled in the art can be made thereto without departing from the present invention.

It should, therefore, be emphasized that the above-described embodiments of the present invention, particularly, any “preferred” embodiments, are possible examples of implementations merely set forth for a clear understanding of the principles for the invention. Many variations and modifications may be made to the above-described embodiment(s) of the invention without substantially departing from the spirit and principles of the invention. All such modifications are intended to be included herein within the scope of this disclosure and the present invention, and protected by the following claims.

What is claimed is:

1. An apparatus for providing multisensory stimuli to induce a relaxation response comprising:
a vest comprising a front section and a rear section wherein the front section includes two side portions and each side portion having a plurality of pockets on the outside for holding a vibrating transducer for providing a vibratory stimulation, and the rear section of the vest having a plurality of pockets on the outside for holding same devices; and,
a control device operatively coupled to one or more said transducer for controlling multisensory stimuli.

2. The apparatus of claim 1, wherein the transducer receives a vibratory control signal from the control device.

3. The apparatus of claim 14, wherein the control device is responsive to biofeedback or neurofeedback sensors to enhance sensory simulation utilizing a variety of visual, audio, temperature, and/or olfactory sensory actuators.

4. The apparatus of claim 2, wherein the vibration control signal is variable.

5. The apparatus of claim 4, wherein vibration acceleration provided by said transducer is variable.

6. The apparatus of claim 5, wherein the vibration control signal and the acceleration are adjusted by the use of a timer or a presetting device.

7. The apparatus of claim 1, wherein the control device includes at least one audio presentation.

8. The apparatus of claim 7, wherein the at least one audio presentation includes a hypnotic suggestion for overcoming a behavioral problem.

9. An apparatus for providing multisensory stimuli to induce a relaxation response comprising:
a mattress cover having a plurality of pockets for holding transducers for providing a vibratory stimulation; and,
a control device for controlling said transducers.

10. The apparatus of claim 9, wherein the transducers receive a vibration control signal from a control device.

11. The apparatus of claim 10, wherein the control device is responsive to biofeedback or neurofeedback sensors to enhance sensory simulation utilizing a variety of visual, audio, temperature, and/or olfactory sensory actuators.

12. The apparatus of claim 10, wherein the vibration control signal is variable.

13. The apparatus of claim 10, wherein vibration acceleration provided by said transducers is variable.

14. The apparatus of claim 13, wherein the vibration control signal and the acceleration are adjusted by the use of a timer or a presetting device.

15. The apparatus of claim 11, wherein the control device includes at least one audio presentation.

16. The apparatus of claim 15, wherein the at least one audio presentation includes a hypnotic suggestion for overcoming a behavioral problem.

17. An apparatus for providing multisensory stimuli to induce a relaxation response comprising:
a mattress having a plurality of cavities containing transducers for providing a vibratory stimulation; and,
a control device for controlling said transducers.

18. The apparatus of claim 17, wherein the transducers receive a vibration control signal from a control device.

19. The apparatus of claim 17, wherein the control device is responsive to biofeedback or neurofeedback sensors to enhance sensory simulation utilizing a variety of visual, audio, temperature, and/or olfactory sensory actuators.

20. The apparatus of claim 18, wherein the vibration control signal is variable.

21. The apparatus of claim 18, wherein vibration acceleration provided by said transducers is variable.

22. The apparatus of claim 21, wherein the control device includes at least one audio presentation.

23. The apparatus of claim 22, wherein the at least one audio presentation includes a hypnotic suggestion for overcoming a behavioral problem.
